SEQUENCE LISTING (6 sequences)

SEQ ID NO: 1 - DNA, 20 bp, Artificial Sequence
  Synthesized primer cMyc forward primer
caccgagtcgtagtcgaggt

SEQ ID NO: 2 - DNA, 20 bp, Artificial Sequence
  Synthesized primer cMyc reverse primer
gctgcttagacgctggattt

SEQ ID NO: 3 - DNA, 19 bp, Artificial Sequence
  Synthesized primer NMyc forward primer
gcgagctgatcctcaaacg

SEQ ID NO: 4 - DNA, 22 bp, Artificial Sequence
  Synthesized primer NMyc reverse primer
cgcctcgctctttatcttcttc

SEQ ID NO: 5 - DNA, 30 bp, Artificial Sequence
  Synthesized primer B2M forward primer
gaatggagagagaattgaaaaagtggagca

SEQ ID NO: 6 - DNA, 22 bp, Artificial Sequence
  Synthesized primer B2M reverse primer
tcacacggcaggcatactcatc

(19) United States
(12) Patent Application Publication
(10) Pub. No.: US 2016/0022708 A1
(43) Pub. Date: Jan. 28, 2016

(54) METHODS AND COMPOSITIONS FOR THE TREATMENT OF GLUTAMINE-ADDICTED CANCERS

(71) Applicant: ST. JUDE CHILDREN'S RESEARCH HOSPITAL, Memphis, TN (US)

(72) Inventor: Kevin Freeman, Germantown, TN (US)

(73) Assignee: ST. JUDE CHILDREN'S RESEARCH HOSPITAL, Memphis, TN (US)

(21) Appl. No.: 14/774,380

(22) PCT Filed: Mar. 13, 2014
(86) PCT No.: PCT/US14/25759

§ 371 Date: Sep. 10, 2015

Related U.S. Application Data
(60) Provisional application No. 61/783,020, filed on Mar. 14, 2013.

(51) Int. Cl. A61K 31/655; A61K 31/635; A61K 31/404; A61K 45/06; A61K 31/167

(52) U.S. Cl. CPC A61K 31/655; A61K 45/06; A61K 31/167; A61K 31/404; A61K 31/635

(57) ABSTRACT

Provided herein are methods for a novel combination therapy for treating a glutamine-addicted cancer in a subject in need thereof, which comprises the administration of a glutaminase antagonist and a pro-apoptotic compound. Specific glutaminase antagonists and pro-apoptotic compounds are provided. In some embodiments, the glutaminase antagonist is 6-diazo-5-oxo-1-norleucine (DON) and the pro-apoptotic compound is a Bcl-2 family member antagonist. In some embodiments, the pro-apoptotic compound is obatoclax mesylate, navitoclax, or fenretinide. In some embodiments, the glutamine-addicted cancer is a cancer in which Myc is deregulated. In some embodiments, the cancer is a pediatric cancer.

### REFERENCE TO A SEQUENCE LISTING SUBMITTED AS A TEXT FILE VIA EFS-WEB

The official copy of the sequence listing is submitted concurrently with the specification as a text file via EFS-Web, in compliance with the American Standard Code for Information Interchange (ASCII), with a file name of 441728seqlist.txt, a creation date of Mar. 11, 2014, and a size of 2 Kb. The sequence listing filed via EFS-Web is part of the specification and is hereby incorporated in its entirety by reference herein.

### FIELD OF THE INVENTION

The present invention relates to the field of cancer therapy, particularly glutamine-addicted cancers.

### BACKGROUND OF THE INVENTION

Glutamine metabolism plays an important role in many cancers. Glutamine is required for bioenergetics, nucleotide biosynthesis, and redox homeostasis in cancer cells. Many cancers become glutamine addicted and require exogenous glutamine for growth and survival. Since glutamine is not an essential amino acid, glutamine metabolism is an attractive therapeutic target.

Myc is a family of transcription factors that, in normal cells, appear to integrate environmental signals in order to modulate a diverse, and sometimes opposing, group of cellular processes, including proliferation, growth, apoptosis, energy metabolism, and differentiation (Eilers (2008) Genes Dev. 22:2755-2766). Myc deregulation occurs frequently in human cancers and has been estimated to contribute to at least 40% of all human cancers. Recently, there has been a resurgent interest in targeting the abnormal metabolism of cancers and understanding the contribution of c-Myc and N-Myc to these metabolic changes.

Myc reportedly coordinates glutamine metabolism by regulating a number of genes that import and utilize glutamine including the glutamine transporters ASCT2 and LAT1, glutaminase 1, and multiple nucleotide biosynthetic genes. c-Myc has been shown to cause glutamine addiction in multiple cancer cell lines, and recently N-Myc has been shown to have a similar effect in neuroblastoma (NB).

Neuroblastoma (NB) and Ewing's sarcoma (EWS) are devastating pediatric cancers that together cause a combined 18% of childhood cancer deaths. NB is a pediatric embryonal malignancy of the developing sympathetic nervous system with gene amplification of MYCN, a c-Myc family member, occurring in ˜20% of NB patients and associating with poor prognosis. EWS is an aggressive malignancy of the bone and soft tissue characterized by chromosomal translocations that result in expression of EWSR1/ETS fusion proteins. The most common fusion found in 85% of EWS is the EWSR1/FLI fusion that targets c-Myc for overexpression and may cooperate with c-Myc in transforming cells. Despite advances in both diagnosis and treatment, the survival rate for patients with the highest stage neuroblastomas (NB) or Ewing's sarcomas (EWS) is still less than 30%.

Therefore, methods for the treatment of cancers comprising glutamine-addicted cancer cells, particularly those associated with Myc deregulation, are needed.

### BRIEF SUMMARY OF THE INVENTION

Methods of treating a subject for a glutamine-addicted cancer, including those cancers associated with Myc deregulation, are provided. The methods comprise combination therapy with a glutaminase antagonist and at least one pro-apoptotic compound. Administering these two agents in combination provides for greater effectiveness than either agent alone, resulting in a positive therapeutic response. In some embodiments, a synergistic therapeutic effect occurs.

The following embodiments are encompassed by the present invention:

1. A method of treating a glutamine-addicted cancer in a subject in need thereof, said method comprising administering to said subject a therapeutically effective amount of a glutaminase antagonist in combination with a therapeutically effective amount of a pro-apoptotic compound.

2. The method of embodiment 1, wherein the glutaminase antagonist is a glutamine analogue.

3. The method of embodiment 2, wherein the glutamine analogue is 6-diazo-5-oxo-1-norleucine (DON), azaserine, azotomycin, or acivicin.

4. The method of embodiment 2, wherein the glutamine analogue is 6-diazo-5-oxo-1-norleucine (DON).

5. The method of embodiment 1, wherein the pro-apoptotic compound is an anti-apoptotic Bcl-2 family member antagonist.

6. The method of embodiment 5, wherein the anti-apoptotic Bcl-2 family member antagonist targets Bcl-2, Bcl-XL, or Mcl-1.

7. The method of embodiment 5, wherein the anti-apoptotic Bcl-2 family member antagonist is selected from the group consisting of obatoclax mesylate (GX15-070), navitoclax (ABT-263), ABT-737, ABT-199, oblimersen sodium, gossypol (AT-101), Apogossypol, HA-14, Antimycin A, and BH₃Is.

8. The method of embodiment 5, wherein the anti-apoptotic Bcl-2 family member antagonist is obatoclax mesylate or navitoclax.

9. The method of embodiment 1, wherein the pro-apoptotic compound comprises fenretinide (FRT; 4-hydroxyphenyl-retinamide).

10. The method of embodiment 1, wherein the glutaminase antagonist is 6-diazo-5-oxo-1-norleucine (DON) and the pro-apoptotic compound is fenretinide, obatoclax mesylate, or navitoclax.

11. A method of treating a glutamine-addicted cancer in a subject in need thereof, said method comprising administering to said subject a therapeutically effective amount of 6-diazo-5-oxo-1-norleucine (DON) and a therapeutically effective amount of fenretinide.

12. A method of treating a glutamine-addicted cancer in a subject in need thereof, said method comprising administering to said subject a therapeutically effective amount of 6-diazo-5-oxo-1-norleucine (DON) and a therapeutically effective amount of obatoclax mesylate.

13. A method of treating a glutamine-addicted cancer in a subject in need thereof, said method comprising administering to said subject a therapeutically effective amount of 6-diazo-5-oxo-1-norleucine (DON) and a therapeutically effective amount of navitoclax.

14. The method of any one of embodiments 1-13, wherein the cancer is associated with Myc deregulation.

15. The method of any one of embodiments 1-14, wherein the cancer overexpresses Myc.

16. The method of embodiment 15, wherein Myc is c-Myc or N-Myc.

17. The method of any one of embodiments 1-16, wherein said cancer is a solid tumor cancer.

18. The method of any one of embodiments 1-17, wherein said cancer is a pediatric cancer.

19. The method of any one of embodiments 1-18, wherein said cancer is selected from the group consisting of acute lymphocytic leukemia, acute myeloid leukemia, ependymoma, Ewing's sarcoma, glioblastoma, medulloblastoma, neuroblastoma, osteosarcoma, rhabdomyosarcoma, rhabdoid cancer, nephroblastoma (Wilm's tumor), hepatocellular carcinoma, esophageal carcinoma, liposarcoma, bladder cancer, gastric cancer, myxofibrosarcoma, colon cancer, kidney cancer, histiosarcoma, ovarian cancer, endometrial carcinoma, lung cancer, and breast cancer.

20. The method of any one of embodiments 1-19, wherein the cancer is selected from a group consisting of neuroblastoma and Ewing's sarcoma.

21. The method of any one of embodiments 1-20, wherein the glutaminase antagonist is administered orally or intravenously.

22. The method of any one of embodiments 1-21, wherein the pro-apoptotic compound is administered orally or intravenously.

23. The method of any one of embodiments 1-22, wherein the glutaminase antagonist and the pro-apoptotic compound are administered simultaneously.

24. The method of any one of embodiments 1-22, wherein the glutaminase antagonist and the pro-apoptotic compound are administered sequentially.

25. The method of any one of embodiments 1-24, wherein administration of the glutaminase antagonist and the pro-apoptotic compound have a synergistic effect.

These and other aspects of the invention are disclosed in more detail in the description of the invention given below.

### BRIEF DESCRIPTION OF THE FIGURES

FIG. 1: DON is effective against NB and Ewing's Sarcoma cell lines with sensitivity to DON correlating to the glutamine sensitivity of the target cell. Cell viability as a percent of control (% Live Cells) is graphed in a dose response curve of 72 hrs DON treatment across a panel of (A) NB and (B) Ewing's Sarcoma cell lines using the immortalized BJ cell line as a control. Data shown are representative of three independent experiments. (C) Western blot analysis of N-Myc and c-Myc expression in a panel of cell lines with β-tubulin as a loading control. (D) QT-PCR of c-Myc and N-Myc expression in a panel of cell lines. (E) Cell viability as a percent of control (% Live Cells) for cell lines either after 72 hrs treatment with either 0 mM glutamine or media containing 100 μM DON. (F) Correlation coefficient of % Live cells-no glutamine plotted versus % Live cells-100 μM DON derived from (E).

FIG. 2: DON significantly inhibits tumor growth in multiple NB and Ewing's sarcoma cell line tumor models. (A-F) The indicated tumor cell line grew to 200 mm³ subQ prior to initiation of treatment. Tumor size is a percent relative to original tumor volume (% RTV). (A-D) Ten mice per group for SK-N-AS and SK-N-MC, and five mice per group for SK-N-Be(2) and SK-ES-1 were treated with DON at 100 mg/kg or water by I.P. twice weekly. P-values, calculated using Student's t test, for each significant data point are given. Weight loss in mice from DON reduced the treatment cohort to 2 mice indicated by (2) at later timepoints. (E and F) Five mice per group were treated with DON at 100 mg/kg, 50 mg/kg or water (Control) twice weekly or 100 mg/kg once weekly (DON 100 mg/kg; 1×/wk) by I.P. injections. The * indicates p<0.01 between control and 50 mg/kg, while ** indicates p<0.01 between control and 100 mg/kg; 1×/wk, Student's t test.

FIG. 3: DON is strongly cytostatic but differently cytotoxic to two NB tumor lines. (A) Schematic of DON treatment and BrdU administration prior to harvesting tumor tissue for immunohistochemistry (IHC). (B and D) IHC of BrdU-labeled cells or cleaved Caspase-3 captured at 200×. (C and E) Quantification of BrdU and Caspase-3 positive cells, respectively, in both SK-N-AS (5 mice per group) and SK-N-BE(2) (5 mice per group for BrdU and 7 mice per group for Caspase-3) subcutaneous tumors. Significance was determined by Student's t test.

FIG. 4: DON causes cell cycle arrest and increased cell death in two NB cell lines. (A-D) SK-N-AS cells and SK-N-BE(2) cells were treated with water (control) or 100 μM DON for 72 hrs and then treated as described below. (A) cells were pulse-labeled with BrdU labeling for cell cycle analysis by FACS. (B) Cells were stained with annexin antibody and propidium iodide (PI) staining prior to FACS analysis. (C and D) in addition to DON or control treatments, cells were treated daily with either 100 μM nucleotide mix (AMP, CMP, GMP, TMP, UMP, and IMP), 2 mM dimethyl-alpha-ketoglutarate (DAK) or 1× sodium pyruvate. * p<0.05 and ** p<0.01 by Student's t test.

FIG. 5: Identification of small molecule inhibitors that increase DON's effectiveness, in vitro. (A) A schematic of the death pathway following glutamine withdrawal in NB cells. The drugs being tested in combination with DON are illustrated where they would impact this death pathway. (B-D) Using a CyQuant assay, DON was tested in combination with the transaminase inhibitor aminooxyacetate (AOA), the glutamate dehydrogenase inhibitor epigallocatechin-3-gallate (EGCG), the synthetic retinoid derivative fenretinide (Fen), or the BCL-2 family agonist obatoclax mesylate (OBX) across a panel of NB and Ewing's sarcoma cell lines using BJ foreskin fibroblasts as a control. Percent live cells was determined by comparison to the no drug condition for each cell line. *p<0.01 by Student's t test.

FIG. 6: DON and ABT-263 show potent combined effects against NB and EWS at clinically achievable concentrations of both drugs. Using a CyQuant assay, DON was tested in combination with the BCL-2 family agonist navitoclax (ABT-263) across a panel of NB and Ewing's sarcoma cell lines. BJ foreskin fibroblasts were used as a control. Percent live cells was determined by comparison to the no drug condition for each cell line. Data is representative of three independent experiments.

FIG. 7: DON inhibits tumor growth in SK-N-BE2 and IMR32 NB cell line tumor models. The indicated tumor cell line grew to 200 mm³ subQ prior to initiation of treatment. Mice with SK-N-BE2 tumors (A) or IMR32 tumors (B) were treated with DON at 50 mg/kg or water (control) twice weekly by I.P. injections. Tumor volume was determined and is shown for individual animals. Tumor size is also depicted as a percent relative to original tumor volume (% RTV). (C) Summary of tumor size for SK-N-FI, SK-N-BE2 and IMR32 cell line tumor models treated with DON at 50 mg/kg or water (control).

### DETAILED DESCRIPTION OF THE INVENTION

The presently disclosed subject matter provides compositions and methods for treating glutamine-addicted cancers. In particular, a combination therapy that effectively inhibits growth and survival and promotes death of glutamine-addicted cancer cells, including glutamine-addicted cancer cells in which Myc is deregulated, is provided. The combination therapy comprises the administration of a therapeutically effective amount of a first agent, a glutaminase antagonist, in combination with a therapeutically effective amount of at least one second agent, a pro-apoptotic compound. This combination therapy finds use in treating a subject for a cancer which comprises glutamine-addicted cells, including cancer in which expression of Myc is deregulated.

Many cancers demonstrate aberrant regulation of glutamine metabolism. The term “glutamine addiction” or “glutamine-addicted” refers to a phenotype in which a cell depends on exogenous glutamine for survival. Glutamine addiction is reviewed in Wise (2010) Trends Biochem. Sci. 35:427-433, herein incorporated by reference in its entirety. The methods of the invention target glutamine-addicted cancers using drugs that interfere with glutamine metabolism in combination with a second agent that increases the effects of the drug that interferes with glutamine metabolism.

Deregulation of Myc family members can lead to glutamine addiction in cancer cells (Levine (2010) Science 330:1340-4; Heiden (2011) Nat. Rev. Drug Discov. 10:671-84). Myc deregulation is one of the most common oncogenic events observed in cancer and is known to drive the progression of many cancers including human lymphomas, neuroblastoma, and small cell lung cancer. “Myc deregulation” refers to the rearrangement, amplification, overexpression and/or translocation of a Myc family member gene. There are at least three members of the Myc family, which are known as c-Myc, N-Myc, and L-Myc.

Therefore, the methods of the invention include a combination therapy for treating a cancer that comprises glutamine-addicted cancer cells in a subject in need thereof. The first agent in the combination therapy is a glutaminase antagonist. A “glutaminase antagonist” is an agent that reduces, inhibits, or otherwise diminishes one or more of the biological activities of the enzyme glutaminase. Such activities of the glutaminase enzyme include the binding of glutamine, glutamate, or various cofactors to the enzyme. That is, a glutaminase antagonist may block binding of the substrate glutamine to glutaminase, inhibit release of the product glutamate from glutaminase, or block cofactor binding and therefore slow the catalytic rate of the enzyme. Antagonism using the glutaminase antagonist does not necessarily indicate a total elimination of the glutaminase activity. Instead, the activity could decrease by a statistically significant amount including, for example, a decrease of at least about 30%, 35%, 40%, 45%, 50%, 55%, 60%, 65%, more preferably 70%, 80%, 85%, and most preferably 90%, 95%, 99%, or 100% of the activity of glutaminase compared to an appropriate control.

Examples of such glutaminase antagonists include 6-diazo-5-oxo-L-norleucine (DON), N-ethylmaleimide (NEM), p-chloromercuriphenylsulfonate (pCMPS), L-2-amino-4-oxo-5-chloropentoic acid, DON plus o-carbamoyl-L-serine, acivicin [(alphaS,5S)-alpha-amino-3-chloro-4,5-dihydro-5-isoxazoleacetic acid], azaserine, azotomycin, palmitoyl coenzyme A (palmitoyl CoA), stearoyl coenzyme A (stearoyl CoA), bromothymol blue, and combinations or derivatives thereof.

In some embodiments the glutaminase antagonist is a “glutamine analogue.” A glutamine analogue is a molecule that is structurally similar to glutamine. Examples of glutamine analogues include 6-diazo-5-oxo-1-norleucine (DON), azaserine, azotomycin, and acivicin. DON (6-diazo-5-oxo-L-norleucine) is a global competitive irreversible inhibitor of glutamine utilizing enzymes and was originally isolated from Streptomyces.

The combination therapy of the invention also comprises administering a therapeutically effective amount of at least one pro-apoptotic compound in combination with the therapeutically effective amount of the glutaminase antagonist. A “pro-apoptotic compound” increases the tendency of a cell to undergo apoptosis. A pro-apoptotic compound can activate a biochemical pathway that results in apoptosis or can interfere with, or block, a biochemical pathway that inhibits apoptosis.

Pro-apoptotic compounds useful in the combination therapy of the present invention target one or more “Bcl-2 family members.” The Bcl-2 family of proteins comprises both “anti-apoptotic Bcl-2 family members” and “pro-apoptotic Bcl-2 family members.” Examples of anti-apoptotic Bcl-2 family members include Bcl-2, Bcl-XL, Bcl-w, Mcl-1, Bfl1/A-1, and Bcl-B. Examples of pro-apoptotic Bcl-2 family members include Bax and Bak. As used herein an “anti-apoptotic Bcl-2 family member antagonist” is an antagonist of the activity of anti-apoptotic Bcl-2 family members, which can thereby promote apoptosis. The anti-apoptotic Bcl-2 family member antagonist targets Bcl-2 or Bcl-XL. Anti-apoptotic Bcl-2 family member antagonists include obatoclax mesylate (GX15-070), navitoclax (ABT-263), ABT-737, oblimersen sodium, gossypol (AT-101), Apogossypol, HA-14, Antimycin A, BH₃Is, and the like. See Kang et al. (2009) Clin Cancer Res. 15:1126-1132 and U.S. Pat. No. 8,362,014, herein incorporated by reference in their entirety.

As indicated, the pro-apoptotic compounds can target pro-apoptotic biochemical pathways. Such a pro-apoptotic pathway includes the activation of the ATF4 transcription factor. Fenretinide [N-(4-hydroxyphenyl)retinamide; 4-HPR] is a pro-apoptotic compound that has been demonstrated to activate ATF4 and promote apoptosis. Fenretinide is a synthetic analogue of all-trans retinoic acid (ATRA) that exhibits cytotoxic activity against a variety of human cancer cell lines in vitro (Delia et al. (1993) Cancer Res. 53:5374-5376; Mariotti et al. (1994) J. Natl. Cancer Inst. 86:1245-1247; Kalemkerian et al. (1995) J. Natl. Cancer Inst. 87:1674-1680; Oridate et al. (1996) Clin. Cancer Res. 2:855-863; O'Donnell et al. (2002) Leukemia 16:902-910); all of which are herein incorporated by reference in their entirety. Fenretinide has also been studied clinically both as a chemopreventive agent in breast (Veronesi et al. (1999) J. Natl. Cancer Inst. 91:1847-1856), bladder (Sabichi et al. (2008) Clin. Cancer Res. 14:335-229) and oral mucosal cancers (Chiesa et al. (2005) Int. J. Cancer 115:625-629), and more recently as a chemotherapeutic agent in pediatric cancers (Garaventa et al. (2003) Clin. Cancer Res. 9:2032-2039; Villablanca et al. (2006) J. Clin. Oncol. 24:3423-3430) and adult cancers (Puduvalli et al. (2004) J. Clin. Oncol. 22:4282-4289; Vaishampayan et al. (2005) Invest. New Drugs 23:179-185; Reynolds et al. (2007) J. Clin. Oncol. 25:18s). Such references are herein incorporated by reference.

In one aspect of the invention the combination therapy comprises the administration of a therapeutically effective amount of DON in combination with a therapeutically effective amount of fenretinide. In another aspect, the combination therapy comprises the administration of a therapeutically effective amount of DON in combination with a therapeutically effective amount of obatoclax mesylate (GX15-070). Yet another aspect of the combination therapy comprises the administration of a therapeutically effective amount of DON in combination with a therapeutically effective amount of navitoclax (ABT-263).

The pro-apoptotic compounds of the invention enhance the effectiveness of DON at clinically relevant concentrations across a panel of cancer cell lines. Thus, the combination therapy is more effective and is able to target additional tumor cells when compared to the effectiveness of either agent alone.

The combination therapy may provide a synergistic improvement in therapeutic efficacy relative to the individual therapeutic agents when administered alone. The term “synergy” is used to describe a combined effect of two or more active agents that is greater than the sum of the individual effects of each respective active agent. Thus, where the combined effect of two or more agents results in “synergistic inhibition” of an activity or process, for example, tumor growth, it is intended that the inhibition of the activity or process is greater than the sum of the inhibitory effects of each respective active agent. The term “synergistic therapeutic effect” therefore refers to a therapeutic effect observed with a combination of two or more therapies wherein the therapeutic effect (as measured by any of a number of parameters, e.g., tumor growth delay) is greater than the sum of the individual therapeutic effects observed with the respective individual therapies.

The combination therapy of the invention affects cancer cell growth, cancer cell survival, and cancer cell death. As used herein, “cell growth” refers to cell proliferation, cell division, or progression through the cell cycle. “Cell survival” refers to the ability of a cell to avoid cell death, including both apoptosis and necrosis. “Cell death” includes both apoptosis and necrosis. Assays for measuring cancer cell growth, survival, and death are known in the art (von Bubnoff et al. (2005) Blood 105:1652-1659; von Bubnoff et al. (2006) Blood 108:1328-1333; Kancha et al. (2009) Clin Cancer Res 15:460-467; von Bubnoff et al. (2009) Cancer Res 69:3032-3041; von Bubnoff et al. (2005) Cell Cycle 4:400-406; each of which is herein incorporated by reference in its entirety) and described elsewhere herein (see Example 1). “Apoptosis” refers to the commonly understood process of programmed cell death carried out by biochemical pathways within a cell.

Any method known in the art can be used to measure the growth rate of a cell or an effect of the disclosed combination therapy on cell survival, including, but not limited to, optical density (OD₆₀₀), CO₂ production, O₂ consumption, assays that measure mitochondrial function, such as those utilizing tetrazolium salts (e.g., MTT, XTT), or other colorimetric reagents (e.g., the WST-1 reagent available from Roche), assays that measure or estimate DNA content, including, but not limited to fluoremetric assays such as those utilizing the fluorescent dye Hoechst 33258, assays that measure or estimate protein content, including, but not limited to, the sulforhodamine B (SRB) assay, manual or automated cell counts (with or without the Trypan Blue stain to distinguish live cells), and clonogenic assays with manual or automated colony counts. Non-limiting examples of assays that can be used to measure levels of apoptosis include, but are not limited to, measurement of DNA fragmentation, caspase activation assays, TUNEL staining, and annexin V staining.

As discussed, the methods disclosed herein provide a glutaminase antagonist and at least one pro-apoptotic compound as a “combination therapy.” “Combination therapy” is herein defined as the application or administration of two or more therapeutic compounds to a subject.

By “subject” is intended mammals, e.g., primates, humans, agricultural and domesticated animals such as, but not limited to, dogs, cats, cattle, horses, pigs, sheep, and the like. In some embodiments, the subject who is being treated is a human. By “human patient” is intended a human subject who is afflicted with, at risk of developing or relapsing with, any disease or condition associated with a cancer.

The term “cancer” refers to the condition in a subject that is characterized by unregulated cell growth, wherein the cancerous cells are capable of local invasion and/or metastasis to noncontiguous sites. As used herein, “cancer cells,” “cancerous cells,” or “tumor cells” refer to the cells that are characterized by this unregulated cell growth and invasive property. “Tumor” (or “tumour”), as used herein, refers to all neoplastic cell growth and proliferation, whether malignant or benign, and all pre-cancerous and cancerous cells and tissues. “Neoplastic,” as used herein, refers to any form of dysregulated or unregulated cell growth, whether malignant or benign, resulting in abnormal growth. Thus, “neoplastic cells” include malignant and benign cells having dysregulated or unregulated cell growth.

In some embodiments, the cancer that is being treated is a solid tumor cancer, which refers to cancers that are characterized by a localized mass of tissue that is capable of locally invaded its surrounding tissues or metastasizing to a noncontiguous site. Solid tumor cancers are distinct from lymphomas and leukemias, which are cancers of the blood cells that typically do not form solid masses of cells.

The combination therapy of the invention is useful for the treatment of glutamine-addicted cancers. By “glutamine-addicted cancer” is intended a cancer that comprises neoplastic cells that are glutamine addicted. Methods of identifying glutamine-addicted cancers are known to those of skill in the art. A non-limiting example is glutamine starvation described by Qing (2012) Cancer Cell 22:631-644. Preferably the cancer cells are primary cancer cells. Cancer cells suspected of being glutamine addicted may be cultured in glutamine free medium for 48 hours. Following glutamine starvation, the number of dead cells are counted. Cell death may be assayed by any method known in the art. Significantly increased cell death compared to cells grown in glutamine-containing media indicates that the cells are glutamine addicted. See also the assays disclosed in the Experimental Section herein below.

Of particular interest is the treatment of glutamine-addicted cancers that are associated with Myc deregulation. By “cancer associated with Myc deregulation” or “Myc-deregulated cancer” is intended a cancer comprising neoplastic cells with rearrangement, amplification, overexpression and/or translocation of a Myc family member gene. Myc family members, including c-Myc, N-Myc, and L-Myc, are known in the art and discussed in Hermeking (2003) Current Cancer Drug Targets 3:163-175. The coding sequences for Myc genes are known in the art. See, for example, Dalla-Favera (1982) PNAS 79:6497-6501, Dalla-Favera (1982) PNAS 79:7824-7827 (c-Myc, GenBank Accession No. NG_{—}007161), Brodeur (1984) Science 224:1121-1124 (N-Myc, GenBank Accession No. NG_{—}007457), and Nau (1995) Nature 318:69-73(L-Myc, GenBank Accession No. AC_{—}000133), herein incorporated by reference in their entirety. Methods of identifying cancers that are associated with Myc deregulation are known in the art and include any assay by which Myc expression and/or gene copy can be determined. Such methods include but are not limited to Western blot, ELISA, fluorescence microscopy, immunohistochemistry, RT-PCR, real-time RT-PCR, and Northern blot, ChIP, and Myc-driven reporter assays. See also the assays disclosed in the Experimental Section herein below.

Myc-deregulated cancers include bladder cancer, breast cancer, colon cancer, gastric cancer, hepatocarcinoma, melanoma, myeloma, neuroblastoma, ovarian cancer, prostate cancer, rhabdomyosarcoma, small cell lung cancer, subungual melanoma, uveal melanoma, Ewing's sarcoma, leukemia, and lymphoma. Myc-deregulated cancers also include pediatric cancer, which is a cancer where the onset or diagnosis of which occurs during the early stages of life prior to full physical maturity (i.e., embryonic, fetal, infancy, pre-pubertal, adolescent). In some embodiments, the pediatric cancer is a pediatric solid tumor cancer. In particular embodiments, the pediatric cancer is a pediatric acute lymphocytic leukemia (e.g., B-cell acute lymphocytic leukemia), acute myeloid leukemia, ependymoma, Ewing's sarcoma, glioblastoma, medulloblastoma, neuroblastoma, osteosarcoma, rhabdomyosarcoma, rhabdoid cancer, or nephroblastoma.

The term “cancer,” however, can encompasses all types of cancers, including, but not limited to, all forms of carcinomas, melanomas, sarcomas, lymphomas and leukemias, including without limitation, cancers of the cardiac system: sarcoma (angiosarcoma, fibrosarcoma, rhabdomyosarcoma, liposarcoma), myxoma, rhabdomyoma, fibroma, lipoma and teratoma; cancers of the lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hanlartoma, inesothelioma; cancers of the gastrointestinal system: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); cancers of the genitourinary tract: kidney (adenocarcinoma, Wilm's tumor [neplrroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); cancers of the liver: hepatoma (hepatocellular carcinoma), cholangiocarcinoma, hepatoblastoma, angiosarcoma, hepatocellular adenoma, hemangioma; cancers of the bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; cancers of the nervous system: skull (osteoma, hemangioma, granuloma, xanthoma, osteitis deformians), meninges (meningioma, meningiosarcoma, gliomatosis), brain (astrocytoma, medulloblastoma, glioma, ependymoma, germinoma [pinealoma], glioblastorna multiform, oligodendroglioma, schwannoma, retinoblastoma, congenital tumors), spinal cord neurofibroma, meningioma, glioma, sarcoma); gynecological cancers: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); hematologic cancers: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma], anaplastic large cell lymphoma (ALCL); skin cancers: malignant melanoma, basal cell carcinoma, squamous cell carcinoma, Karposi's sarcoma, moles dysplastic nevi, lipoma, angioma, dermatofibroma, keloids, psoriasis; and cancers of the adrenal glands: neuroblastoma. The combination therapy disclosed herein finds use in treating any of the forgoing cancers where the cancer comprises glutamine-addicted neoplastic cells.

The combination therapy disclosed herein finds use in treating a subject for a glutamine-addicted cancer. In some embodiments, the glutamine-addicted cancer is associated with Myc deregulation. By “treating” is intended the combination therapy provides for a positive therapeutic response with respect the cancer undergoing treatment. By “positive therapeutic response” is intended an improvement in the disease, and/or an improvement in the symptoms associated with the disease, as a result of the therapeutic activity of the combination therapy. That is, an anti-proliferative effect, the prevention of further tumor outgrowths, a reduction in tumor size, a reduction in the number of neoplastic cells, and/or a decrease in one or more symptoms associated with the cancer for which the subject is undergoing treatment can be observed. Thus, for example, a positive therapeutic response would refer to one or more of the following improvements in the disease: (1) a reduction in tumor size; (2) a reduction in the number of neoplastic cells; (3) an increase in neoplastic cell death; (4) inhibition of neoplastic cell survival; (4) inhibition (i.e., slowing to some extent, preferably halting) of tumor growth; (5) inhibition (i.e., slowing to some extent, preferably halting) of neoplastic cell infiltration into peripheral organs; (6) inhibition (i.e., slowing to some extent, preferably halting) of tumor metastasis; (7) the prevention of further tumor outgrowths; (8) an increased patient survival rate; and (9) some relief from one or more symptoms associated with the cancer.

Positive therapeutic responses in any given cancer can be determined by standardized response criteria specific to that cancer. Tumor response can be assessed for changes in tumor morphology (i.e., overall tumor burden, tumor size, and the like) using screening techniques such as magnetic resonance imaging (MRI) scan, x-radiographic imaging, computed tomographic (CT) scan, bone scan imaging, endoscopy, and tumor biopsy sampling including bone marrow aspiration (BMA) and counting of tumor cells in the circulation. In addition to these positive therapeutic responses, the subject undergoing therapy may experience the beneficial effect of an improvement in the symptoms associated with the cancer.

The combination therapy methods of the invention involve the use of therapeutically effective amounts of a glutaminase antagonist and a pro-apoptotic compound. The terms “therapeutically effective dose,” “therapeutically effective amount,” or “effective amount” are intended to mean an amount of the glutaminase antagonist or pro-apoptotic compound that, when administered as a part of a combination therapy comprising at least these two agents, brings about a positive therapeutic response with respect to treatment of a glutamine-addicted cancer in a subject.

It is understood that appropriate doses of glutaminase antagonist and the pro-apoptotic compound depend upon a number of factors within the knowledge of the ordinarily skilled physician, veterinarian, or researcher. The dose(s) of these agents will vary, for example, depending upon the age, weight, disease progression, and condition of the subject being treated, further depending upon the route by which the agents are to be administered and the effect that the practitioner desires the therapeutic agents to have on the cancer being treated. Exemplary doses include milligram or microgram amounts of these therapeutic agents per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). It is furthermore understood that appropriate doses of these therapeutic agents depend upon the potency of the respective agents with respect to the expression or activity to be modulated. Such appropriate doses may be determined using the assays described herein. A physician, veterinarian, or researcher may, for example, prescribe a relatively low dose at first, subsequently increasing the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, the specific drug combination, and the like.

Generally, for a single dose of DON the amount to be administered may be in the range from about 50 to about 600 mg/m² (milligrams per body surface of the recipient), from about 50-600 mg/m², from about 150-550 mg/m², from about 150-450 mg/m², 150-350 mg/m², from about 200-400 mg/m², from about 250-400 mg/m², or from about 300-400 mg/m², based on the recipient. Further, for a single dose of DON, the amount to be administered may be about 150 mg/m², about 225 mg/m², about 300 mg/m², about 375 mg/m², about 450 mg/m², about 520 mg/m², about 550 mg/m², about 575 mg/m², or about 600 mg/m². Methods and pharmaceutical compositions for administering DON have been described in Sullivan (1988) Cancer Chemother Pharmacol 21:78-84, herein incorporated by reference in its entirety. A dose of DON may be administered in the specified amount preferably four times a week to once every two weeks, more preferably three times a week to once a week, more preferably twice a week every two weeks, and even more preferably twice a week. In some embodiments, the dose of DON of about 150 mg/m², about 225 mg/m², about 300 mg/m², about 375 mg/m², about 450 mg/m², about 520 mg/m², about 550 mg/m², about 575 mg/m², or about 600 mg/m² may be administered once a week. In other embodiments, the dose of DON of about 150 mg/m², about 225 mg/m², about 300 mg/m², about 375 mg/m², about 450 mg/m², about 520 mg/m², about 550 mg/m², about 575 mg/m², or about 600 mg/m² may be administered twice a week.

Generally the amount of the pro-apoptotic compound will be administered in ranges established for each. For a single dose of fenretinide the amount to be administered may be in the range of about 300-2500 mg/m². Further, for a single dose of fenretinide, the amount to be administered may be about 300 mg/m², about 400 mg/m², about 500 mg/m², about 600 mg/m², about 700 mg/m², about 800 mg/m², about 900 mg/m², about 1000 mg/m², about 1100 mg/m², about 1200 mg/m², about 1300 mg/m², about 1400 mg/m², about 1500 mg/m², about 1600 mg/m², about 1700 mg/m², about 1800 mg/m², about 1900 mg/m², about 2000 mg/m², about 2200 mg/m², about 2300 mg/m², about 2400 mg/m², or about 2500 mg/m².

Methods and pharmaceutical compositions for administering fenretinide have been described in Veronesi et al. (1999) J. Natl. Cancer Inst. 91:1847-1856; Sabichi et al. (2008) Clin. Cancer Res. 14:335-229; Chiesa et al. (2005) Int. J. Cancer 115:625-629; Garaventa et al. (2003) Clin. Cancer Res. 9:2032-2039; Villablanca et al. (2006) J. Clin. Oncol. 24:3423-3430; Puduvalli et al. (2004) J. Clin. Oncol. 22:4282-4289; Vaishampayan et al. (2005) Invest. New Drugs 23:179-185; Reynolds et al. (2007) J. Clin. Oncol. 25:18s; the contents of each of which are herein incorporated by reference in their entirety.

For a single dose of navitoclax (ABT-263) the amount to be administered may be determined as follows. Dosage amounts for this agent are expressed herein as free base equivalent amounts unless the context requires otherwise. Typically, a unit dose (the amount administered at a single time), which can be administered at an appropriate frequency, e.g., twice daily to once weekly, is about 10 to about 1,000 mg. Where frequency of administration is once daily (q.d.), unit dose and daily dose are the same thing. For example, the unit dose of navitoclax in a composition of the invention can be about 25 to about 1,000 mg, more typically about 50 to about 500 mg, for example about 50, about 100, about 150, about 200, about 250, about 300, about 350, about 400, about 450 or about 500 mg. Where the composition is prepared as a discrete dosage form such as a tablet or capsule, a unit dose can be deliverable in a single dosage form or a small plurality of dosage forms, most typically 1 to about 10 dosage forms. For example, suitable doses of navitoclax are administered at an average dosage interval of about 3 hours to about 7 days, for example about 8 hours to about 3 days, or about 12 hours to about 2 days. In most cases a once-daily (q.d.) administration regimen is suitable.

A daily dosage amount effective to maintain a therapeutically effective navitoclax plasma level may be about 50 to about 500 mg. In most cases a suitable daily dosage amount is about 200 to about 400 mg. For example, the daily dosage amount can be for example about 50, about 100, about 150, about 200, about 250, about 300, about 350, about 400, about 450 or about 500 mg.

An average dosage interval effective to maintain a therapeutically effective navitoclax plasma level is, according to the present embodiment, about 3 hours to about 7 days. In most cases a suitable average dosage interval is about 8 hours to about 3 days, or about 12 hours to about 2 days. A once-daily (q.d.) administration regimen is often suitable.

Further, for a single dose of navitoclax, the amount to be administered may be about 10 mg/day, about 20 mg/day, about 30 mg/day, about 130 mg/day, about 225 mg/day, about 325 mg/day, about 425 mg/day, or about 475 mg/day. Further, navitoclax may be administered to a subject on a dosing cycle. The cycle may be a 21-day dose cycle. In a first dose cycle, navitoclax is administers on days 1 to 14 of the 21-day dose cycle. In a second dose cycle, navitoclax is administered on days 1 to 21 of a 21-day dose cycle. Methods and pharmaceutical compositions for administering navitoclax have been described in Rudin (2012) Clin. Cancer Res. 18:3163-3169; Tse (2008) Cancer Res. 68:3420-3428; Gandhi (2011) J. Clin. Oncol. 29:909-916; US2010/0278921 and are herein incorporated by reference in entirety.

For a single dose of obatoclax, the amount to be administered may be about 1-100 mg/m² (milligrams per body surface of the recipient), 1-40 mg/m², 3-30 mg/m², 3-14 mg/m². Further, the dose of obatoclax may be from about 1 mg/m², from about 2 mg/m², from about 3 mg/m², from about 4 mg/m², from about 5 mg/m², from about 6 mg/m², from about 7 mg/m², from about 8 mg/m², from about 9 mg/m², from about 10 mg/m², from about 11 mg/m², from about 12 mg/m², from about 13 mg/m², from about 14 mg/m², from about 15 mg/m², from about 16 mg/m², from about 17 mg/m², from about 18 mg/m², from about 19 mg/m², from about 20 mg/m², from about 25 mg/m², from about 30 mg/m², from about 35 mg/m², from about 40 mg/m², from about 45 mg/m², or from about 50 mg/m².

Further, obatoclax may be administered to a subject on a dosing cycle. The dosing cycle may be the administration of obatoclax 2 times per week, 1 time per week, once every 2 weeks, or once every 3 weeks. Obatoclax may be administered, for example, using a 1-hour infusion duration or a 3-hour infusion duration. The treatment can be continued for between 1 and 40 cycles. Further the treatment can be continued for up to 3 cycles, up to 4 cycles, up to 5 cycles, up to 6 cycles, up to 7 cycles, up to 8 cycles, up to 9 cycles, up to 10 cycles, up to 15 cycles, up to 20 cycles, up to 25 cycles, or up to 30 cycles. Methods and pharmaceutical compositions for administering obatoclax have been described by O'Brien (2009) Blood 113:299-305 and are herein incorporated by reference in entirety.

If the active ingredients are solids, the active ingredients can be made into solid pharmaceutical preparations by the usual processes, for example by mixing the two active ingredients together and converting the mixture for example into tablets with the usual carriers and excipients. It is also possible, however, to supply the two active ingredients separately in one commercial packaging unit, wherein the packaging unit comprises both active ingredients but in separate pharmaceutical formulations.

In a preferred embodiment according to the invention, the active ingredients are supplied in the form of injection or infusion solutions. The injection or infusion solutions can be optionally applied separately from each other.

In the case of a parenteral dosage form, the active ingredients can be present in the original form, possibly together with the usual pharmaceutical excipients (for example in the lyophilized form), and then reconstituted or solubilized by the addition of pharmaceutically customary injection or infusion media.

The pharmaceutical preparations are applied in liquid or solid form in the case of enteral or parenteral application. All the usual application forms are possible here, for example tablets, capsules, sugarcoated tablets, syrups, solutions and suspensions. The injection medium is preferably water, containing the usual additives employed in injection solutions, such as stabilizers, solubilizers and buffers. Additives of this kind include tartrate and citrate buffers, ethanol, complexants such as ethylenediaminetetraacetic acid and its non-toxic salts, as well as high-molecular polymers, such as liquid polyethylene oxide for viscosity adjustment. The liquid carriers for injection solutions must be sterile, and they are preferably supplied in ampules. Solid carriers are for example starch, lactose, silica, higher molecular fatty acids such as stearic acid, gelatine, agar, calcium phosphate, magnesium stearate, animal and vegetable fats, and high-molecular solid polymers like polyethylene glycols. Preparations suitable for oral administration may contain flavors and sweeteners, if desired.

Accordingly, the concentrations used can also be varied during a cycle, depending e.g., on the occurrence of unexpected recipient-specific side effects. The individual administration units of the glutaminase antagonist and/or the pro-apoptotic compound can be varied during the cycle according to the recipient and any undesirable side effects.

The combination therapy according to the invention can be administered in the form of a fixed combination, i.e. as a single pharmaceutical formulation comprising the glutaminase antagonist and the pro-apoptotic compound, or else it can be used in a free combination, where the glutaminase antagonist and the pro-apoptotic compound are applied in separate pharmaceutical formulations, simultaneously or successively. When there are two administration units A) and B), they can be formulated independently both as a liquid, both as a solid, or one as a solid and one as a liquid.

In one embodiment the glutaminase antagonist (also referred to as “the glutaminase antagonist agent”) and the pro-apoptotic compound (also referred to as “the pro-apoptotic agent”) may be administered to the patient at the same time or at separate times. When administered concomitantly, the glutaminase antagonist agent may be administered to the patient at exactly the same time as the pro-apoptotic agent (i.e., the two agents are administered simultaneously). Alternatively, the glutaminase antagonist agent may be administered to the patient at approximately the same time as the pro-apoptotic agent (i.e., the two agents are not administered at precisely the same time), e.g., during the same visit to a physician or other healthcare professional. The simultaneous administration may be repeated as needed.

In other embodiments, the glutaminase antagonist agent and the pro-apoptotic agent are not administered to the patient at the same time, but are administered sequentially (consecutively), in either order. In these embodiments, the methods of the invention may comprise administering a first cycle of pro-apoptotic agent to the patient before a first dose of the glutaminase antagonist is administered to the patient. Alternatively, the methods may comprise administering a first cycle of the pro-apoptotic agent to the patient after a first dose of the glutaminase antagonist is administered to the patient. In embodiments where the glutaminase antagonist agent and the pro-apoptotic agent are administered sequentially, the agents may be administered in such a way that both agents exert a therapeutic effect on the patient at the same time (i.e., the periods in which each therapy is effective may overlap) although this is not essential.

The agents of the invention may be provided in one or more pharmaceutical compositions and thus may be administered along with a pharmaceutically acceptable carrier. As used herein the term “pharmaceutically acceptable carrier” includes solvents, dispersion media, antibacterial and antifungal agents, isotonic agents, and the like, compatible with pharmaceutical administration. Supplementary active compounds also can be incorporated into the composition(s).

A pharmaceutical composition of the invention is formulated to be compatible with its intended route of administration. Examples of routes of administration include intravenous and oral administration.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor® EL (BASF; Parsippany, N.J.), or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride, in the composition. Prolonged absorption of the injectable compositions can be brought about by including in the composition an agent that delays absorption, for example, aluminum monostearate and gelatin.

Sterile injectable solutions can be prepared by incorporating the active agent(s) of the invention in the required amount in an appropriate solvent with one or a combination of ingredients enumerated above, as required, followed by filtered sterilization. Generally, dispersions are prepared by incorporating the active agent(s) into a sterile vehicle that contains a basic dispersion medium and the required other ingredients from those enumerated above. In the case of sterile powders for the preparation of sterile injectable solutions, the preferred methods of preparation are vacuum drying and freeze-drying, which yields a powder of the active agent(s) plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active agent(s) can be incorporated with excipients and used in the form of tablets, troches, or capsules. Oral compositions can also be prepared using a fluid carrier for use as a mouthwash, wherein the agent(s) in the fluid carrier is (are) applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth, or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

It will be understood by one of skill in the art that the treatment modalities described herein may be used alone or in conjunction with other therapeutic modalities (i.e., as adjuvant therapy), including, but not limited to, surgical therapy, radiotherapy, chemotherapy (e.g., with any chemotherapeutic agent well known in the art) or immunotherapy.

It is to be noted that the term “a” or “an” entity refers to one or more of that entity; for example, “a cell” is understood to represent one or more cells. As such, the terms “a” (or “an”), “one or more,” and “at least one” can be used interchangeably herein.

Throughout this specification and the claims, the words “comprise,” “comprises,” and “comprising” are used in a non-exclusive sense, except where the context requires otherwise.

As used herein, the term “about,” when referring to a value is meant to encompass variations of, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the presently disclosed subject matter be limited to the specific values recited when defining a range.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

The following examples are offered by way of illustration and not by way of limitation.

### EXPERIMENTAL

### Example 1

### DON is an Effective Chemotherapeutic In Vitro with Sensitivity to DON Correlating with Glutamine Addiction

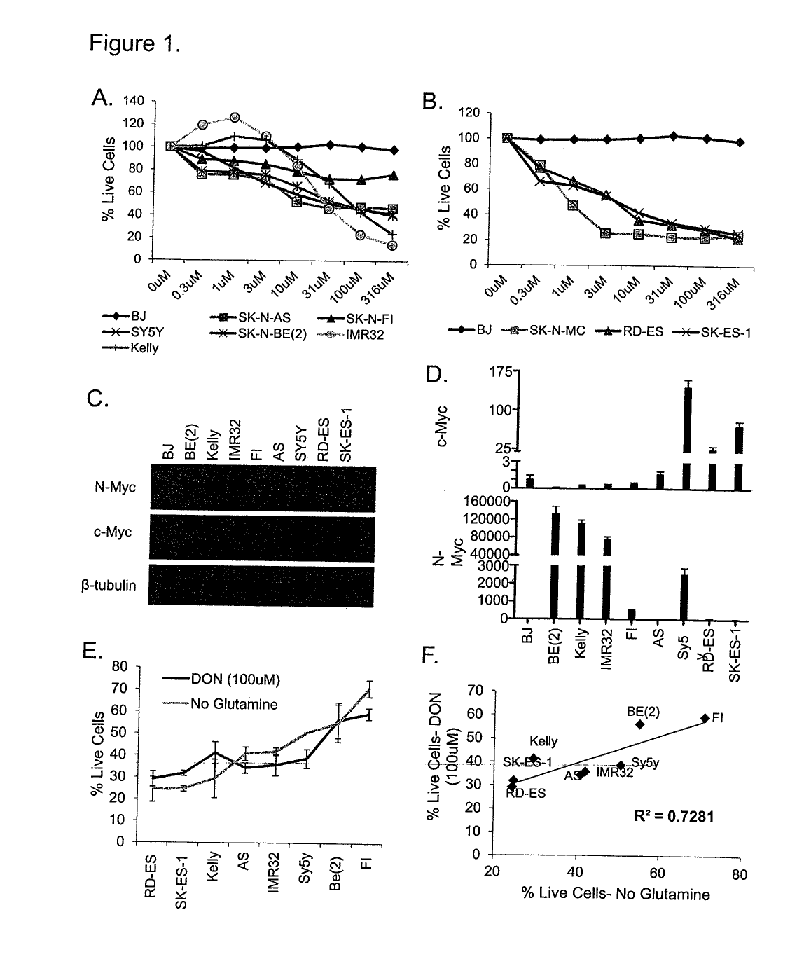

To survey the effects of DON on neuroblastomas and Ewing's sarcomas, DON was tested on six neuroblastoma (SK-N-AS, SK-N-BE(2), SK-N-FI, IMR32, Kelly, and SH-SySy) and three Ewing's sarcoma cell lines (SK-N-MC, RD-ES and SK-ES-1) and a control immortalized foreskin fibroblast cell line (BJ). Using the CyQuant viability assay, the respective sensitivities of each cell line to DON were determined after 72 hrs of treatment in a universal media (EMEM, 10% FBS, 0.5 mM glutamine) containing physiological levels of glutamine and glucose. With the exception of the control BJ cells, all cell lines showed some sensitivity to DON. SK-N-FI cells were the most resistant with only a 20% reduction in cell numbers while multiple cell lines showed greater than 60% loss in cell numbers (FIGS. 1A and 1B). Previous pharmacokinetics data suggests that the DON concentrations in these assays are physiologically achievable in children. More strikingly, all three Ewing's sarcoma cell lines were especially sensitive to DON with an 80% loss in cell viability (FIG. 1B).

Based on Myc's known role in glutamine addiction, whether sensitivity of neuroblastoma and Ewing's sarcoma cell lines to DON corresponded with glutamine addiction and Myc expression levels was assessed. c-Myc and N-Myc expression was measured by western blot analysis (FIG. 1C) and quantitative PCR (FIG. 1D). To determine the degree of glutamine addiction in the neuroblastoma and Ewing's Sarcoma cell lines cells were cultured in EMEM+10% dialyzed FBS with or without 2 mM glutamine and the percentage of live cells after 72 hours of glutamine deprivation was measured by CyQuant analysis (FIG. 1E). An association between increased N-Myc expression and increased glutamine addiction was observed, with Kelly cells expressing more N-Myc and being more glutamine addicted than IMR32 which were expressing more N-Myc and were more glutamine addicted than SK-N-BE(2) (FIGS. 1C and 1E). A weaker relationship was observed between c-Myc expression levels and glutamine addiction. Though the neuroblastoma cell line Sy5y shows high c-Myc expression (FIG. 1C), it is more resistant to glutamine deprivation than the c-Myc expressing Ewing's Sarcoma cell lines RD-ES and SK-ES-1 or the neuroblastoma cell line SK-N-AS, which does not express Myc (FIG. 1C). However, when sensitivity to glutamine deprivation against the percentage of live cells after 72 hours of treatment with 100 μM DON (FIG. 1E) was tested, a strong correlation (R²=0.728) between glutamine addiction and DON sensitivity (FIG. 1F) was found, reconfirming DON as a global inhibitor of glutamine metabolism.

### DON Severely Affects Tumor Growth

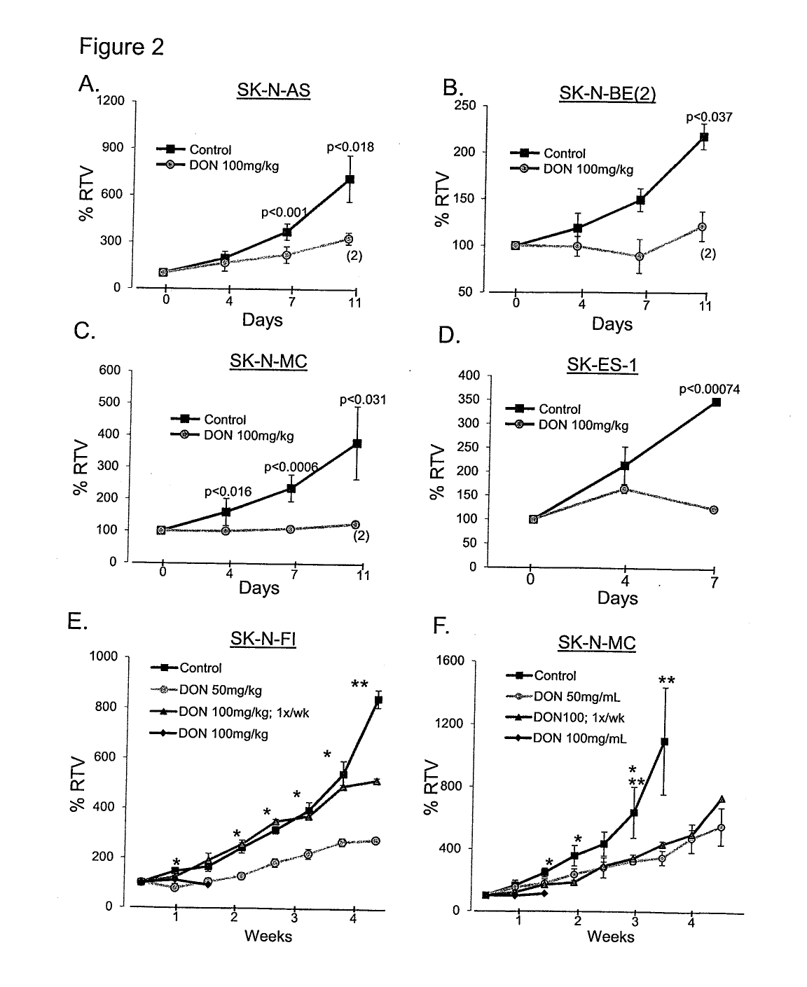

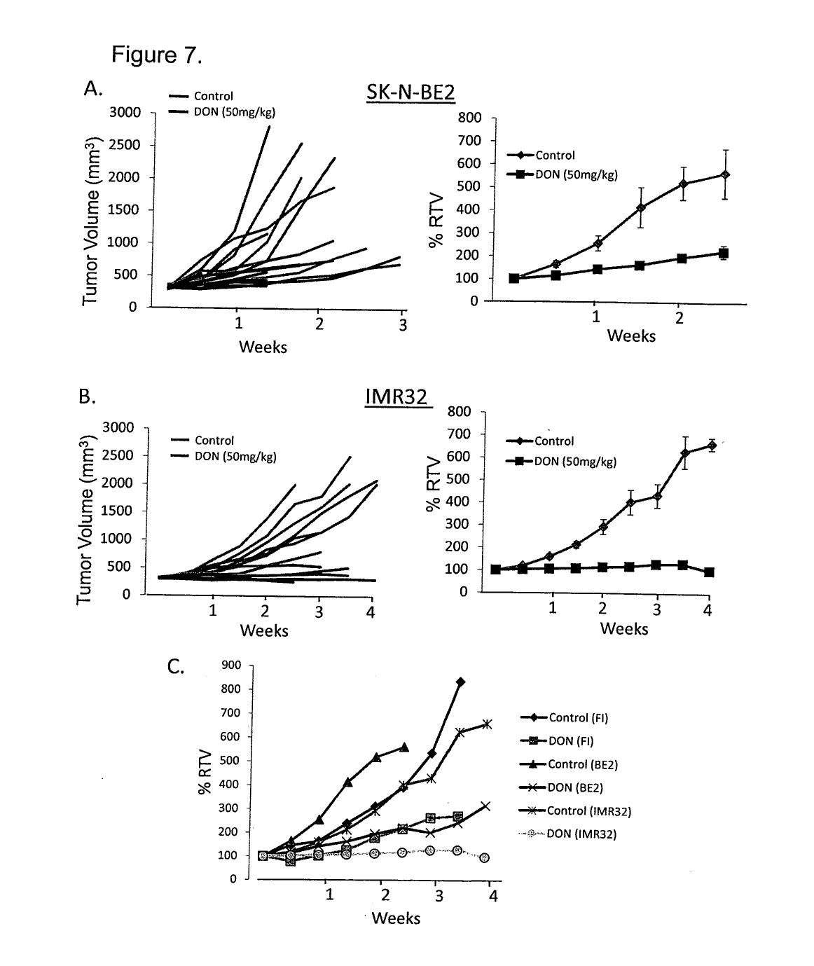

DON's effects on NB and EWS tumors in vivo were assessed. Subcutaneous tumors for the neuroblastoma cell lines SK-N-AS and SK-N-BE(2) as well as the Ewing's Sarcoma cell lines SK-N-MC and SK-ES-1 were grown to at least 200 mm³ and were then randomized into either DON or control treatment groups. Mice were then treated with 100 mg/kg of DON or water control twice a week by intraperitoneal (I.P.) injection, equivalent to a twice a week dose of 300 mg/m² in children. Previous work has shown that a dose as high as 520 mg/m² was safely achievable in children. At the 100 mg/kg dose, DON greatly reduced growth of both the low Myc expressing neuroblastoma SK-N-AS tumors and high N-Myc expressing SK-N-BE(2) tumors (FIGS. 2A and 2B). DON treatment also dramatically blocked growth of the high c-Myc expressing EWS tumors, SK-N-MC and SK-ES-1 (FIGS. 2C and 2D). These results are especially impressive considering the exponential growth of the control tumors, which doubled every 4 days for SK-N-AS and SK-ES-1 tumors, and every 4-7 days for SK-N-MC and SK-N-BE(2). However, DON at these doses caused weight loss in the mice that limited the extent of treatment. To attempt to extend the course of treatment, mice either were treated with half the dose at 50 mg/kg twice a week or with a once weekly injection of 100 mg/kg (1×/wk). The effectiveness of the original drug regimen was compared to the two new treatment schedules using mice with subcutaneous tumors from the most DON-resistant (SK-N-FI) and the most DON-sensitive (SK-N-MC) cell line in vitro. This experiment would allow for the determination whether the in vitro data was predictive of differences in response to DON in vivo. Both of the new drug regimens allowed a significant extension in length of treatment from 11 days to greater than 24 days. However, only the 50 mg/kg dose was effective against both tumor types. In contrast, the 100 mg/kg (1×/wk) dose showed significant differences between SK-N-FI and SK-N-MC, which corresponded to the differing sensitivity of these cell lines to DON in vitro (FIGS. 2E and 2F). In addition, the SK-N-BE2 and IMR32 neuroblastoma cell lines were tested at the 50 mg/kg dose in mice with subcutaneous tumors. The 50 mg/kg dose was effective against both tumor types (FIG. 7 A-C). In all cell lines tested, in vivo DON treatment significantly reduced growth of tumors, including SK-N-FI tumors, suggesting that DON could be broadly efficacious against NB and EWS.

### DON is Strongly Cytostatic but Differentially Cytotoxic to Two Neuroblastoma Tumor Lines.

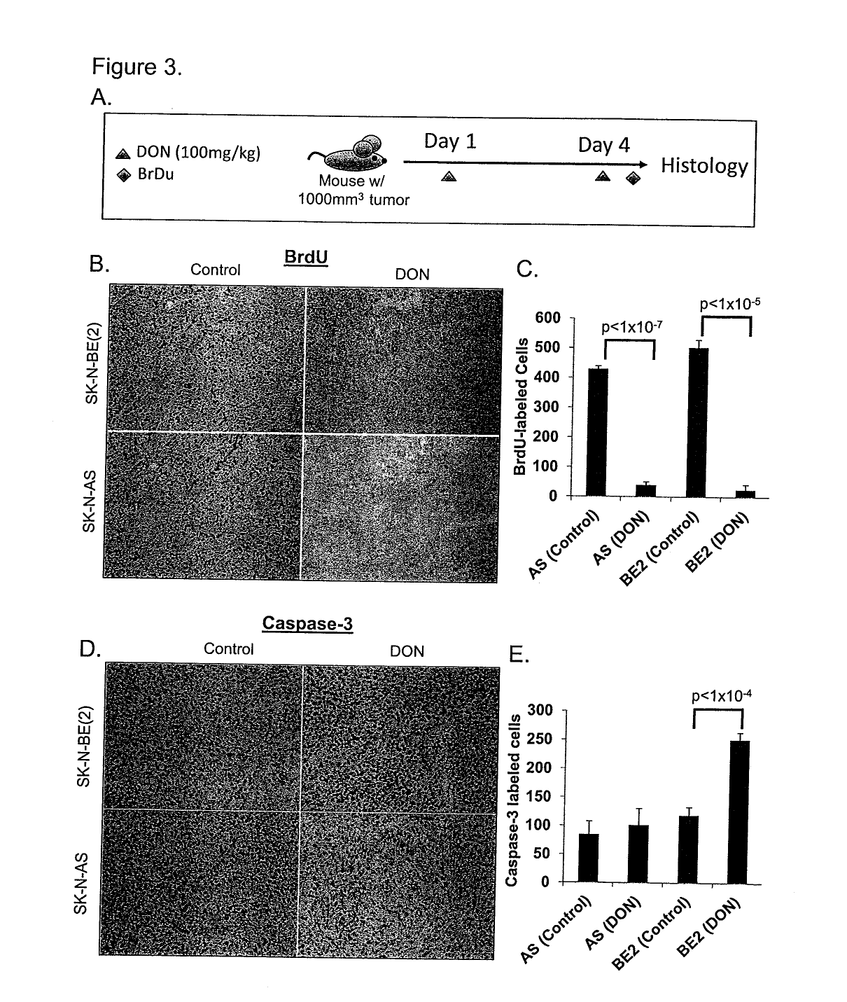

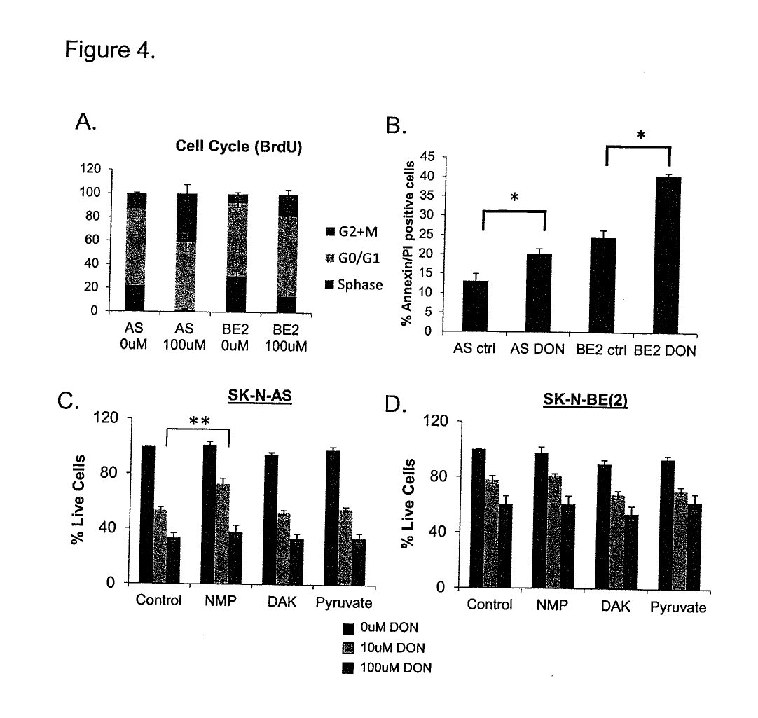

DON greatly reduced or blocked tumor growth when administered to mice at 100 mg/kg twice weekly. To investigate the effects of DON on proliferation and cell death in vivo, additional NB tumor studies using SK-N-AS and SK-N-BE(2) cells were set up. Tumors in each cohort were allowed to grow to ˜1000 mm³ before they underwent one round of DON treatment at 100 mg/kg of DON twice a week. On day 4, the second dose of DON was given 6-hrs prior and BrdU injected 2 hours prior to harvesting the tumors for histology (FIG. 3A). Both SK-N-BE(2) and SK-N-AS showed a >90% reduction in BrdU incorporation after treatment with DON (FIGS. 3B and 3C). Using cleavage of caspase-3 as a marker for apoptosis, SK-N-BE(2) tumors showed a greater than 2-fold increase in caspase-3 cleavage when treated with DON, while SK-N-AS tumors showed no difference (FIGS. 3D and E). To confirm the in vivo results, DON was tested on both cell lines in vitro and found by cell cycle analysis using BrdU labeling that there was a reduced entry into the S-phase of the cell cycle in both SK-N-AS and SK-N-BE(2) cell lines (FIG. 4A). Both cell lines also showed an increase in cell death, with SK-N-BE(2) showing more marked effects (FIG. 4B).

Previous work by Yuneva et al., used a Myc-ER inducible cell line to determine that replenishment of the CAC by glutaminolysis was critical for Myc-induced cell death following glutamine deprivation. Another report showed a delayed S-phase transit in K-ras transformed cells grown in low glutamine, which was reversed with addition of exogenous nucleotides during the first 24-hrs. However, the repeat addition of nucleotides only partially reversed (25%) the cell count when cells were grown in low-glutamine over 96-hrs. The authors suggested that loss of glutamine dependent pathways other than nucleotide synthesis is likely responsible for the primary decrease in cell number. Therefore, whether cell permeable exogenous factors downstream of DON targets could potentially reverse the effects of DON was examined. The results demonstrate that nucleotides could partially reverse the effects in SK-N-AS at 10 μM but not at 100 μM DON, and nucleotides could not overcome the effects of DON on SK-N-BE(2) at either dose (FIGS. 4C and 4D). Further, the addition of exogenous pyruvate or dimethyl-ketoglutarate, a cell permeable variant of the CAC intermediate alpha-ketoglutarate, was tested. However, no effects on either cell line were observed. The results in SK-N-AS cells suggest that the effects of DON at 10 μM are partly due to inhibition of nucleotide synthesis. However, either multiple glutamine utilizing pathways are responsible for the effects of DON at 100 μM, or a pathway that cannot be replenished with exogenous factors, such as the hexosamine pathway, is responsible for the observed results.

Identification of Small Molecule Inhibitors that Increase DON's Efficacy

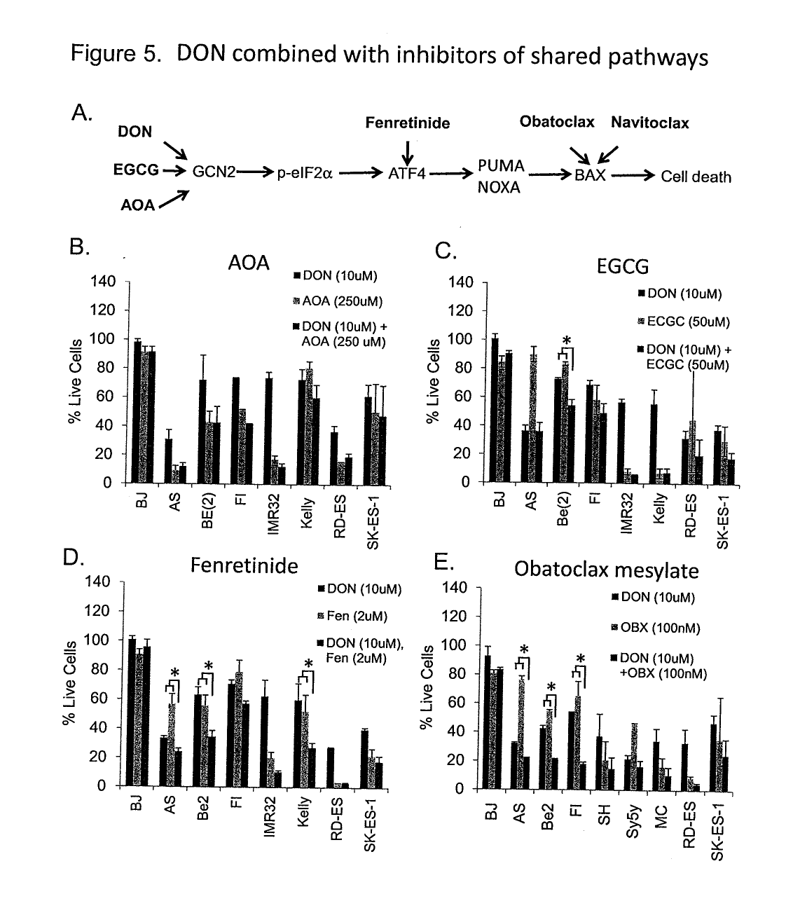

The IHC results show DON to be strongly cytostatic and modestly cytotoxic. However, interference with glutamine metabolism is known to cause cellular stress that leads to apoptosis. A recent publication described many of the relevant signaling factors that cause apoptosis after glutamine withdrawal in neuroblastoma cell lines. To summarize their findings, glutamine deprivation led to increased transcription and translation of the ATF4 transcription factor. ATF4 in turn increased expression of the pro-apoptotic factors PUMA and NOXA leading to cell death via BAX (FIG. 5A). Therefore, combining DON with other factors that target glutamine metabolism or that augment key components of the death signaling pathway downstream of glutamine starvation should increase the effects of DON on both neuroblastoma and Ewing's sarcoma cell lines.

Glutaminase, which is targeted by DON, is the rate-limiting enzyme of glutaminolysis catalyzing the conversion of glutamine into glutamate. Glutamate is then turned into the CAC intermediate alpha-ketoglutarate by either glutamate dehydrogenase (GDH) or glutamine transaminases. The green tea polyphenol epigallocatechin-3-gallate (EGCG) is an in vitro inhibitor of GDH while aminooxyacetate (AOA) is a non-specific competitive inhibitor of amino acid transaminases including glutamine transaminases. As inhibitors of glutaminolysis, EGCG and AOA were recently shown to be toxic to neuroblastoma cells and EGCG impaired growth of subcutaneous neuroblastoma Kelly tumors. The effects of EGCG on tumor growth were enhanced by fenretinide, a retinoic acid derivative that is proapoptotic and has been reported to cause death through activation of ATF4 (FIG. 5A). The effects of DON in combination with AOA, EGCG, or fenretinide were tested and the best results were observed when DON was used in conjunction with fenretinide (FIG. 5B-D). As the apoptotic effect of glutamine deprivation goes through the proapoptotic BAX protein and cancers often overexpress anti-apoptotic Bcl-2 family members, DON was tested in combination with obatoclax mesylate, a small molecule Bcl-2 family inhibitor currently in a phase I pediatric clinical trial. DON combined with the pan-Bcl-2 antagonist obatoclax mesylate significantly enhanced the effects of DON (FIG. 5E). Specifically the results with the combination of DON and obatoclax mesylate against SK-N-FI cells were especially promising as this cell line is resistant to many of the drugs tested. Also the data demonstrate that combining DON with chemotherapeutics that act further downstream in the death-signaling pathway, such as fenretinide and obatoclax mesylate, was more effective then combining DON with agents that acted further upstream, like AOA and EGCG.

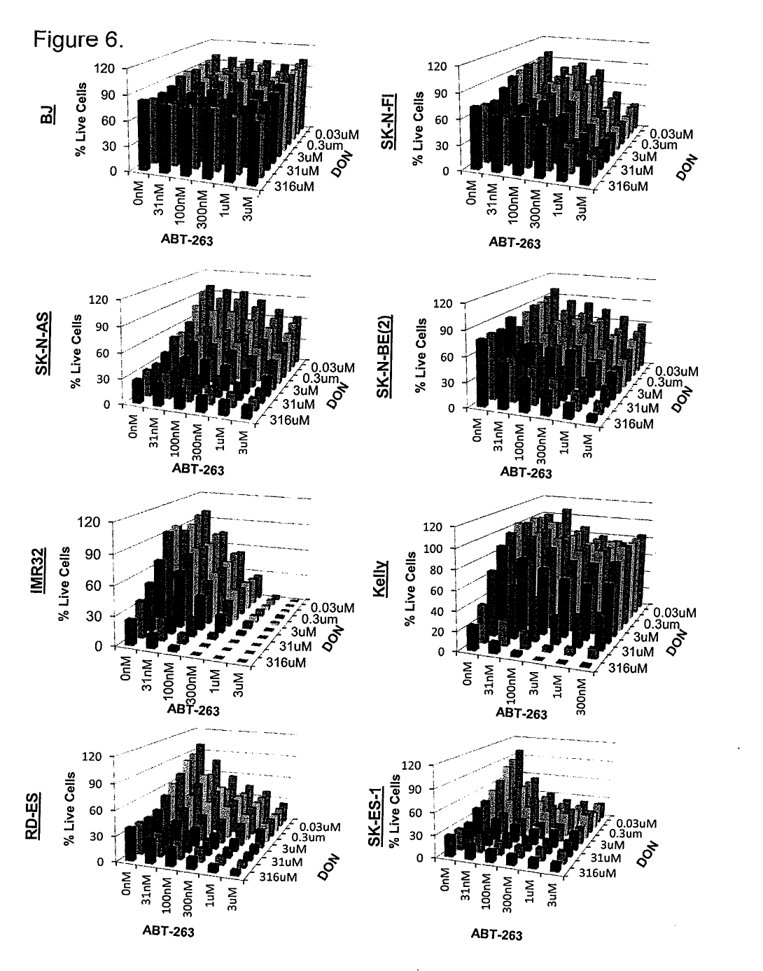

DON was tested with another commercially available anti-Bcl-2 inhibitor, ABT-263 (Navitoclax). ABT-263 is orally bioavailable, safe and well-tolerated with thrombocytopenia as its major adverse side effect. DON with ABT-263 showed strong cooperative effects at clinically achievable concentration of both drugs across a broad range of doses in virtually all of the cell lines tested (FIG. 6). The greatest effect of DON and ABT-263 was demonstrated in cell lines with the highest N-Myc expression (IMR32 and Kelly) or c-Myc expression (RD-ES and SK-ES-1) (FIG. 1C and FIG. 6).

A statistical test procedure to determine synergy of the combination treatment of DON and ABT-263 was performed on the cell viability data for the various cell lines. By using the Loewe additive drug combination reference model (see, for example, Novick, S. J., 2013, Stat. in Med. 32:5145-55), concentration combinations for the SK—N-BE2 cell line and the Kelly cell line were identified as having significant synergism. The results of this analysis are shown below in Tables 1 and 2. An additive effect was identified for all other concentration combinations tested in the SK-N-BE2 and Kelly cell lines (not shown). An additive effect of the DON and ABT-263 combination was identified for the SK-ES-1, RD-ES, SK-N-AS, SK-N-FI and IMR32 cell lines (not shown).

The dramatic effects demonstrated when DON was combined with Bcl-2 inhibitors indicates that DON-induced apoptosis depends on the balance of pro- and anti-apoptotic factors in the cell. These results suggest targeting glutamine metabolism while reducing the threshold for apoptosis is a very promising treatment strategy for neuroblastoma, Ewing's sarcomas, and possibly other glutamine-addicted malignancies.

TABLE 1
Statistical analysis of synergy for DON and ABT-263
combination on cell viability of SK-N-BE2 cells.

| ABT-263 | DON | | Standard | Raw p | Adjusted | |
|---|---|---|---|---|---|---|
| (nM) | (μM) | N | Error | value | p value | Decision |
| 0.1 | 100 | 6 | 0.2006 | 3.00E−04 | 0.0118 | Synergy |
| 0.1 | 316 | 6 | 0.2154 | 0 | 0.0018 | Synergy |
| 0.316 | 31.6 | 6 | 0.2069 | 4.00E−04 | 0.0182 | Synergy |
| 0.316 | 100 | 6 | 0.2069 | 0 | 2.00E−04 | Synergy |
| 0.316 | 316 | 6 | 0.2069 | 0 | 0 | Synergy |
| 1 | 31.6 | 6 | 0.2188 | 5.00E−04 | 0.0228 | Synergy |
| 1 | 100 | 6 | 0.2188 | 0 | 3.00E−04 | Synergy |
| 1 | 316 | 6 | 0.2188 | 0 | 0 | Synergy |
| 3.166 | 100 | 6 | 0.2412 | 1.00E−04 | 0.0027 | Synergy |
| 3.166 | 316 | 6 | 0.2412 | 0 | 5.00E−04 | Synergy |

TABLE 2
Statistical analysis of synergy for DON and ABT-
263 combination on cell viability of Kelly cells.

| ABT-263 | DON | | Standard | Raw p | Adjusted | |
|---|---|---|---|---|---|---|
| (nM) | (μM) | N | Error | value | p value | Decision |
| 1 | 100 | 6 | 0.1157 | 0.0011 | 0.0442 | Synergy |
| 3.166 | 31.6 | 6 | 0.1215 | 6.00E−04 | 0.0256 | Synergy |
| 3.166 | 100 | 6 | 0.1157 | 4.00E−04 | 0.0172 | Synergy |

### DISCUSSION

An idea gaining prominence in the field of cancer metabolism is that the oncogenes that transform cells also regulate and create a reliance on the metabolic pathways that facilitate cell growth. After glucose, glutamine is the next most catabolized nutrient in cancer cells, yet glutamine's importance and regulation in cancer is just starting to be understood. This study shows it is crucially important to understand glutamine metabolism in pediatric cancers as it is a promising avenue for discovering novel therapeutic targets. Other studies have shown that apoptosis due to glutamine withdrawal is due to loss of glutaminolysis. Based on the literature, both EGCG and AOA should target the second step of glutaminolysis and both were effective as single agents when they were tested against some of the more glutamine-addicted cell lines such as IMR32, Kelly, RD-ES and SK-ES-1 (FIGS. 5 B and C). If other antagonists that specifically target glutaminolysis are found they might make promising chemotherapeutic agents.

Of more immediate clinical interest is identifying already available compounds that can be further developed for treating patients. DON (6-diazo-5-oxo-L-norleucine) globally interferes with glutamine metabolism and this study has shown it has broad effects against a variety of NB and EWS cell lines both in vitro and in vivo. Because DON's effects are mostly cytostatic in vivo, DON was screened with drugs that were likely to increase its cytotoxic effects and three clinically relevant compounds, fenretinide, obatoclax mesylate and navitoclax, were identified. Importantly, DON, fenretinide and obatoclax mesylate have been tested as individual therapeutic agents in pediatric clinical trials. Fenretinide was most effective in patients with high serum concentrations of the drug and has been extensively tested in children with neuroblastoma. Though fenretinide's utility is limited by poor bioavailability, multiple groups have identified divergent and promising strategies to solve this problem. Combining fenretinide with other compounds like DON could lower the concentrations necessary for its effect in vivo. Though ABT-263 (Navitoclax) has not been tested in children, it has undergone extensive phase I and phase II clinical trials in adults and was well tolerated. DON, fenretinide and ABT-263 all have broad ranges of drug concentrations that are effective on all the cancer cell lines tested in this study while being non-toxic to BJ cells.

It was shown in vitro as single agents DON, obatoclax mesylate, and ABT-263 show some efficacy but were more potent in combination. Though DON in combination with ABT-263 looks very promising for NB, this drug combination looks especially effective against Ewing's sarcomas. Though no Bcl-2 antagonists are currently FDA approved, there is a large class of these compounds currently moving through clinical trials increasing the likelihood of future FDA approval. Furthermore, continued testing of other Bcl-2 antagonists should identify additional promising drugs capable of enhancing the effects of DON.

The results with DON support mounting evidence in the cancer metabolism field that targeting glutamine metabolism is a promising therapeutic strategy and identifying other inhibitors of glutamine metabolism could yield promising new therapies for pediatric cancers. However, DON is currently the most clinically advanced anti-glutamine metabolite even though it was last tested in children thirty years ago.

### Materials and Methods

### Cell Culture

BJ fibroblasts were purchased from ATCC, and were maintained in EMEM media supplemented with 10% FBS, 1% Penicillin/Streptomycin, 2 mM L-glutamine, 1 mM sodium pyruvate, and 1.5 g/L sodium bicarbonate. Kelly cells were purchased from Sigma-Aldrich, and were maintained in RPMI-1640 media supplemented with 10% FBS, 1% Penicillin/Streptomycin, and 2 mM L-glutamine. All remaining cell lines were obtained from Dr. Michael Dyer and Dr. John Sandoval (St. Jude Children's Research Hospital), and maintained as described below: IMR-32 and SK-N-MC cells were cultured in EMEM supplemented with 10% FBS, 1% Penicillin/Streptomycin, and 2 mM L-glutamine. SK-N-FI and SK-N-AS cells were cultured in DMEM (1 g/L glucose) supplemented with 10% FBS, 1% Penicillin/Streptomycin, 1.5 g/L Sodium Bicarbonate, and 4 mM L-glutamine. SK-SY5Y and SK-N-BE(2) cells were cultured in 1:1 EMEM/Ham's F-12 media supplemented with 10% FBS, 1% Penicillin/Streptomycin, and 2 mM L-glutamine. RD-ES cells were cultured in RPMI-1640 supplemented with 15% FBS, 1% Penicillin/Streptomycin, and 2 mM L-glutamine. SK-ES-1 cells were cultured in McCoy's 5A media supplemented with 10% FBS, 1% Penicillin/Streptomycin, and 2 mM L-glutamine. All cell lines were grown at 37° C. in a humidified atmosphere with 5% CO₂. Identity of all cell lines was verified by both STR analysis and karyotyping at the St. Jude Hartwell Center for Bioinformatics and Biotechnology and the St. Jude Cytogenetics Lab, respectively. Additionally, all cell lines were PCR tested and shown to be mycoplasma free.

qRT-PCR

RNA was isolated from sub-confluent cell lines using the RNeasy Mini kit (Qiagen), and cDNA was synthesized with the High-Capacity cDNA Reverse Transcription Kit (Applied Biosystems). Quantitative real-time PCR was performed on a 7900HT Real Time PCR Machine (Applied Biosystems) using Fast SYBR green master mix (Applied Biosystems) according to manufacturer's protocol. Primers used for amplification include the following: cMyc Fwd 3′-CACCGAGTCGTAGTCGAGGT-5′ (SEQ ID NO:1), cMyc Rev 3′-GCTGCTTAGACGCTGGATTT-5′ (SEQ ID NO:2), NMyc Fwd 3′-GCGAGCTGATCCTCAAACG-5′ (SEQ ID NO:3), NMyc Rev 3′-CGCCTCGCTCTTTATCTTCTTC-5′ (SEQ ID NO:4), B2M Fwd 3′-GAATGGAGAGAGAATTGAAAAAGTGGAGCA-5′ (SEQ ID NO:5), and B2M Rev 3′-TCACACGGCAGGCATACTCATC-5′ (SEQ ID NO:6). All primer pairs were validated using serial 1:10 cDNA dilutions and shown to have equivalent amplification efficiency, therefore Ct values were normalized to B2M and the AACt method was used to determine relative expression levels.

### Western Blotting and Antibodies

MPER (Mammalian Protein Extraction Reagent, Pierce ThermoScientific) was used to make protein lysates from sub-confluent cultures of all cell lines using manufacturer's protocol. Lysis buffer was supplemented with Halt Protease and Phosphatase Inhibitor Cocktail (Pierce/ThermoScientific) and protein concentrations were measured with the BCA Assay (Pierce/ThermoScientific). Lysate volumes containing equal protein were loaded on polyacrylamide gels (Bio-Rad) then transferred to 0.2 μm PVDF membranes (Invitrogen) using a Semi-Dry Transfer system (Bio-Rad). Membranes were blocked in 5% milk/PBST for one hour, followed by primary antibody incubation overnight at 4° C. Antibodies used include the following: cMyc (1:200, R&D #AF3696), NMyc (1:1000, Cell Signaling Technology #9405), β-tubulin (1:1000, Cell Signaling Technology #2146), anti-goat-HRP (1:2000), and anti-rabbit-HRP (1:2000, Cell Signaling Technology #7074S). Membranes were developed using ECL Plus (GE Healthcare/Amersham) and exposed to HyBlot CL autoradiography film.

### Annexin

To measure early apoptosis, cells were plated in EMEM media containing 0.5 mM L-glutamine, 10% FBS, and 1% Penicillin/Streptomycin, then treated+/−100 μM DON for 72 hours. Both adherent and floating cells in the media were collected at harvest, and 3×10⁵ cells were then stained with Annexin-V-FITC antibody for 15 minutes at room temperature. Cells were then counterstained with propidium iodide (9.1 μM final concentration) and filtered through a 40 μm nylon mesh. Immediately after staining, samples were analyzed by the Flow Cytometry and Cell Sorting Shared Resource facility at St. Jude Children's Research Hospital.

### BrdU Labeling

In vitro cell cultures were pulse-labeled with 10 μM BrdU (BD Biosciences) for 90 minutes at 37° C., and then fixed and stained with Anti-BrdU-FITC antibody and 7-AAD (BD Biosciences, #559619), according to manufacturer's protocol. Stained samples were then analyzed by the Flow Cytometry and Cell Sorting Shared Resource facility at St. Jude Children's Research Hospital.

### Cyquant Assay

6-diazo-5-oxo-L-norleucine (Sigma Aldrich, St. Louis, Mo.) and epigallocatechin-3-gallate (Sigma Aldrich, St. Louis, Mo.) were solubilized in water, aminooxyacetate (Sigma Aldrich, St. Louis, Mo.), obatoclax mesylate (Selleckchem,), and ABT-263 (Sellekchem) were dissolved in 100% dimethyl sulfoxide (DMSO). In a 96-well tissue culture plate, 5×10⁴ cells were plated in 100 ul of media containing EMEM, 10% FBS and 0.5 mM glutamine per well. Four hours after plating drugs were diluted to a 2× concentration in plating media and added to wells. Plates were incubated for 72-hrs and then submitted to CyQuant Cell Direct Proliferation Assay (Life Technologies) according to manufacturer's instructions and read with a Synergy HT Multi-mode microplate reader (Biotek).

### Animal Experiments

For subcutaneous tumor experiments, cells were injected into the hind flank of either 6-8 week old athymic (nu/nu) mice (Charles River) or into Rag2^{-/-}; gamma c^{-/-} mice (kindly provided by Dr. Shannon McKinney-Freeman). No difference in tumor growth kinetics was observed between different strains. SK-N-AS, SK-N-MC, and SK-ES-1 cells were injected at 2×10⁶ cells per mouse in 100 μl total volume. SK-N-BE(2) cells were injected in a 1:1 mix of cells and Matrigel (BD Biosciences) at a final concentration of 2×10⁶ cells per mouse. Tumor size was measured by digital caliper, and tumor volume was calculated using the formula 1/2*(length*width²). When tumors reached an average size of 200 mm³ mice were randomized into treatment groups. On the day of treatment, mice were weighed then given an intraperitoneal (i.p.) injection of either 100 mg/kg DON, 50 mg/kg or water control. All animal experiments were performed in accordance with the guidelines established by IACUC.

### BrdU Tumor Experiments

For in vivo labeling of proliferating cells, mice with subcutaneous SK-N-AS or SK-N-BE(2) tumors (1000 mm³) were treated with DON and injected with BrdU (BD Biosciences). Mice were given an intraperitoneal (i.p.) injection of 100 mg/kg DON on day 1 and again on day 4. Four hours after the second dose of DON, mice were injected i.p. with 150 μl of 10 mg/ml BrdU. Tumor tissue was harvested 2 hours after addition of BrdU and samples were fixed in 10% formalin. Paraffin-embedded tissue sections were then stained for BrdU incorporation by the St. Jude Veterinary Pathology Core.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the foregoing list of embodiments and appended claims.

1. A method of treating a glutamine-addicted cancer in a subject in need thereof, said method comprising administering to a subject in need thereof a therapeutically effective amount of 6-diazo-5-oxo-1-norleucine (DON) and a therapeutically effective amount of a pro-apoptotic compound.

2. The method of claim 1, wherein the pro-apoptotic compound is an anti-apoptotic Bcl-2 family member antagonist.

3. The method of claim 2, wherein the anti-apoptotic Bcl-2 family member antagonist targets Bcl-2 or Bcl-XL.

4. The method of claim 2, wherein the Bcl-2 family member antagonist is obatoclax mesylate or navitoclax.

5. The method of claim 1, wherein the pro-apoptotic compound is fenretinide (FRT; 4-hydroxyphenyl-retinamide).

6. The method of claim 1, wherein the pro-apoptotic compound is fenretinide, obatoclax mesylate, or navitoclax.

7. The method of claim 1, wherein the cancer is associated with Myc deregulation.

8. The method of claim 1, wherein the cancer overexpresses Myc.

9. The method of claim 7, wherein Myc is c-Myc or N-Myc.

10. The method of claim 1, wherein the cancer is neuroblastoma or Ewing's sarcoma.

11. The method of claim 1, wherein 6-diazo-5-oxo-1-norleucine (DON) is administered orally or intravenously.

12. The method of claim 1, wherein the pro-apoptotic compound is administered orally or intravenously.

13. The method of claim 1, wherein 6-diazo-5-oxo-1-norleucine (DON) and the pro-apoptotic compound are administered simultaneously.

14. The method of claim 1, wherein 6-diazo-5-oxo-1-norleucine (DON) and the pro-apoptotic compound are administered sequentially.
